(19) United States
(12) Patent Application Publication
(10) Pub. No.: US 2004/0022877 A1
(43) Pub. Date: Feb. 5, 2004

(54) Cardiovascular health enhancement with soy fortified orange juice compositions

(76) Inventors: Nancy Green, Bradenton, FL (US); Richard N. McArdle, Bradenton, FL (US); Carla McGill, Sarasota, FL (US); Renee Mellican, Bradenton, FL (US); Kristin Parshall, Bradenton, FL (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 10/209,216

(22) Filed: Jul. 30, 2002

(51) Int. Cl. A61K 35/78; A61K 33/00; A61K 33/26; A61K 31/525; A61K 31/355; A61K 31/375

(52) U.S. Cl. 424/736; 424/757; 424/646; 424/722; 514/474; 514/458; 514/251

(57) ABSTRACT

A method is provided for administering to an individual a soy fortified orange juice composition which includes soy protein that readily dissolves in orange juice and does not unacceptably negatively impact sensory, mouth feel and viscosity attributes of the orange juice component. When administered daily, these compositions enhance cardiovascular health indicators in the individual. The cardiovascular health indicators include cholesterol levels and blood pressure readings for the individual so treated.

### BACKGROUND OF THE INVENTION

This invention generally relates to therapeutic procedures for positively modifying one or more health indicator levels in mammals. More particularly, the invention relates to modifying cholesterol levels and/or blood pressure levels in a manner which is believed to result in health benefits, especially cardiovascular health benefits. The effects achieved in accordance with the present invention are consistent with reduced risks of cardiovascular disease.

It is generally accepted that an important component in maintaining a profile for good cardiovascular health is the maintenance of desirable cholesterol levels and/or blood pressure readings. Currently it is generally accepted that an individual should avoid certain elevated plasma total cholesterol levels. Two major components of plasma cholesterol are low-density lipoprotein (LDL) cholesterol and high-density lipoprotein (HDL) cholesterol. More particularly, LDL cholesterol levels should be maintained below an acceptable level, while high HDL cholesterol levels are considered to contribute cardiovascular health. It is generally accepted that a decreased LDL to HDL cholesterol ratio is an advantageous goal for those whose cholesterol ratio is higher than desirable levels.

A typically accepted dietary intervention regimen for altering blood cholesterol concentrations is to take measures in order to reduce LDL cholesterol levels. Generally, such dietary intervention does not enjoy the ability of increasing HDL cholesterol and/or reducing blood pressure levels. It will be appreciated that, if a viable dietary intervention program were available for reducing LDL cholesterol levels, increasing HDL cholesterol and/or for decreasing the ratio of LDL to HDL cholesterol levels, while also reducing blood pressure, considerable potential benefits would be provided.

Previous epidemiological studies suggested that high dietary intake of fruit and vegetables is associated with reduced risk of coronary heart disease. See, for example, Bors W, Heller W, Michel C, Saran M. Flavonoids as antioxidants: determination of radical scavenging efficiencies. Method Enzymol 1990; 186:343-55. Dietary flavonoids have been proposed to exert the cardioprotective action mainly as inhibitors of LDL oxidation and platelet aggregation. Cook N C, Samman S. Flavonoids—chemistry, metabolism, cardioprotective effects, and dietary sources. J Nutr Biochem 1996; 7:66-76. Some flavonoids, especially those from soybeans, consisting mainly of the isoflavone genistein, have also been suggested to reduce hypercholesterol. Anthony M S, Clarkson T B, Hughes C L Jr., Morgan T M, Burke G L. Soybean isoflavones improve cardiovascular risk factors without affecting the reproductive system of peripubertal Rhesus monkeys. J Nutr 1996; 126:43-50.

In addition, numerous studies have demonstrated various beneficial effects of several vitamins that are abundant in fruits and vegetables. The vitamins C, E and betacarotene have suggested to act mainly as antioxidants. Charleux J L. Beta-carotene, vitamin C, and vitamin E: the protective micronutrients. Nutr Rev 1996; 54:S109-S114. Folic acid and natural folate present at high levels in citrus fruit and in many green vegetables also have been shown to reduce plasma levels of homocystine/homocysteine, an intermediate in methionine metabolism, implicated as a risk factor in cardiovascular disease. Jacques P F, Selhub J, Bostom A G, Wilson P W F, Rosenberg I H. The effect of folic acid fortification on plasma folate and total homocysteine concentrations. N Enql J Med 1999; 340:1449-54. Brouwer I A, Dusseldorp M V, West C E, Meyboom S, Thomas C M G, Duran M, van het Hof K H, Eskes T K A B, Hautvast J G A J, Steegers-Theunissen R P M. Dietary folate from vegetables and citrus fruit decreases plasma homocysteine concentrations in humans in a dietary controlled trial. J Nutr 1999; 129:1135-9. All literature publications and patent publications referenced herein are incorporated by reference hereinto.

Dietary orange juices are recognized as rich sources of components, including phytochemicals such as flavonoids, as well as known essential human nutrients such as folate and vitamin C. High concentrations of folate, potassium present in orange juices could contribute to their cardioprotective action by reducing plasma homocystine/homocysteine, high concentrations of vitamin C could decrease susceptibility of lipoproteins to oxidation, and potassium is linked to reduced risk of high blood pressure and hypertension.

Dietary soy protein is recognized as helping to reduce the risk of coronary heart disease. Current U.S. Food and Drug Administration regulations permit a health claim for reduced risk of coronary heart disease for foods containing at least 6.25 grams of soy protein per reference amount, typically a serving amount. For beverages, for example, this can be stated as at least 6.25 grams of soy protein per 8 ounce serving of beverage.

Included in typically recognized risk factors for coronary heart disease are an elevated blood pressure level, hypertension, an elevated total serum or blood LDL-cholesterol level, and an elevated LDL to HDL serum cholesterol ratio. Reducing risk factors such as these can provide cardiovascular health enhancement to many in the general population.

Information available regarding the potential cardioprotective effects of dietary orange juice in humans include a study conducted in young normocholesterolemic men, where intake of orange juice reduced lipoprotein oxidation, presumably due to high content of vitamin C, but did not change plasma lipid profile. Harats D, Chevion S, Nahir M, Norman Y, Sagee O, Berry E M. Citrus fruit supplementation reduces lipoprotein oxidation in young men ingesting a diet high in saturated fat: presumptive evidence for an interaction between vitamins C and E in vivo. Am J Clin Nutr 1998; 67:240-5.

Another trial, in which unspecified doses of citrus fruit and green vegetables were added to a diet to increase natural dietary folate, showed that this intervention significantly increased plasma content of folate and reduced plasma content of homocystine/homocysteine in healthy subjects. Brouwer et al., supra.

Another study recognized that orange juices play a role in cholesterol health enhancement regarding HDL increases and decreases in the LDL to HDL serum cholesterol ratio. This is reported in Kurowska E M, Spence J D, Jordan J, et al., HDL-cholesterol-raising effect of orange juice in subjects with hypercholesterolemia, Am J Clin Nutr, 2000; 72(5):1095-1100, and is the subject of European Patent Application No. 00991718.8 and PCT/US00/41784, Modification of Cholesterol with Citrus., McGill and Green.

Information regarding blood pressure lowering by orange juice compositions is found in Sprecher D L, Foody J M, Acevedo M, McGill C, et al., Orange juice and blood pressure reduction—a pilot study (the orange JUICE study), J Am Coll Cardiology 2002; 39S:254A (abstract). Potassium, present in citrus juices, can reduce hypertension and stroke risks, as discussed in Ascherio A, Rimm E B, et al., Intake of potassium, magnesium, calcium, and fiber and risk of stroke among U.S. men; Circulation; 1998:1198-1104.

Beneficial changes in blood pressure of a population can have marked effects on cardiovascular disease. It has been estimated that a decrease of just 2 mm Hg in systolic blood pressure will reduce annual stroke rates by 6% and coronary artery disease by 4%. Hypertension Primer, The Essentials of High Blood Pressure, second edition. Izzo J L and Black H R editors. American Heart Association. Dallas, Tex., 1999.

### SUMMARY OF THE INVENTION

In accordance with the present invention, compositions are provided combining orange juice and soy protein having certain characteristics that facilitate soy availability and stability within the juice, which compositions produce beneficial changes which enhance cardiovascular health. The compositions beneficially change cholesterol levels when ingested. HDL levels are increased. LDL levels can be decreased. Also typical of the present invention is a decrease in the LDL to HDL serum cholesterol ratio. Beneficial blood pressure reductions are achieved. Administration of this beneficial combination is enhanced, facilitated and enabled by selecting a soy protein source which has high solubility in orange juice, low color impact, very low bean sensory notes, and no sensorally perceptible grittiness.

It is accordingly a general object of the present invention to modify bloodstream cholesterol concentrations and/or achieve blood pressure reduction through administering orange juice and soy in a single composition.

An aspect of this invention is providing a method for increasing HDL cholesterol levels in mammals, particularly in humans.

Another aspect of this invention is providing a method for decreasing LDL cholesterol levels in mammals, particularly in humans.

Another aspect of the present invention is providing an improved method for decreasing the LDL to HDL cholesterol ratio in the bloodstream of a living being.

Another aspect of this invention is providing an improved method for adjusting serum cholesterol levels by administering dosages of a combined orange juice and soy protein beverage in which the soy protein is suspended within the juice for extended time periods.

Another aspect of this invention is providing a process for serum cholesterol modification and/or blood pressure reduction by administering dosages of soy fortified orange beverages having sensory characteristics which approximate or equal those of orange beverages which are not soy fortified.

Another aspect of the present invention is the lowering of coronary heart disease risk factors by ongoing administration of soy fortified orange juices.

Another aspect of this invention is to administer health enhancing levels of soy fortified orange juice for lowering coronary heart disease risk factors.

These and other objects, aspects, features and advantages of the present invention will be apparent from and clearly understood through a consideration of the following detailed description.

### DESCRIPTION OF THE PREFERRED EMBODIMENTS

Treatment methods according to the present invention embody the use of orange juices. The invention finds special use in orange juice compositions which are of the not from concentrate type. By the orange component of the treatment compositions, the treatment methods administer phytochemicals of the type which naturally occur within round oranges. These advantageously include one or more of phytochemicals selected from the group consisting monoterpenes, terpenes, and flavonoids. Soy proteins provide naturally occurring components and phytochemicals which contribute to the beneficial aspects of the treatment regimen of the invention.

The treatment method compositions and their components are administered at a dosage level which is effective in achieving one or more of the cardiovascular health enhancements. These include decreasing LDL serum cholesterol levels, increasing HDL serum cholesterol levels, decreasing the LDL to HDL serum cholesterol ratio and/or reducing the systolic and/or diastolic blood pressure readings for the subject being treated.

Included in the treatment procedure is the administration of phytochemicals from different components of oranges, including the juice, juice sacks, pulp, peel, seeds and the like. The phytochemicals in accordance with the invention are the ones which absorb into the bloodstream so as to come into contact with components thereof, such as serum cholesterol and/or so as to affect the vascular system so as to lower blood pressure. Soy protein is incorporated in a form which is readily soluble in whole orange juices, most advantageously not from concentrate juice products. The treatment compositions are pasteurized and containerized in accordance with good manufacturing practices and are administered as noted herein.

These soy fortified orange juices and their attendant phytochemicals and beneficial components are administered at concentrations which achieve the principal effects of the present invention as generally discussed herein. A typical administration dosage can be expressed in terms of ounces of soy fortified orange juice administered daily to an individual. When the individual is a human being, 8 to 10 ounces are administered daily, preferably about 15 ounces. For most adults of average size and weight, it is especially preferred that the amount of soy fortified orange juice be at a daily dosage of at least two standard beverage serving sizes, or of about 16 ounces daily. In some instances, enhanced benefits are realized at levels of about 20 ounces daily, or at a level of at least three standard beverage serving sizes, that is of about 24 ounces daily. When especially high soy protein levels are used, these administration levels can be lower.

The treatment is most conveniently administered by ingestion of whole soy fortified orange juice. However constituted, the treatment compositions administered according to the invention include one or more phytochemicals at the levels providing the beneficial serum cholesterol modifications and/or blood pressure reductions which are characteristic of the invention.

Orange juices and soy proteins are rich sources of beneficial components, phytochemicals and essential human nutrients. An example of an essential nutrient in orange juices is vitamin C, known to decrease the susceptibility of lipoproteins to oxidation. Another is potassium, which is linked to reduced risk of hypertension. Vitamin E, when added to citrus juices, contributes to cardiovascular health enhancement. Other components of the juice itself are folates, believed to effect reduction of plasma homocysteine/homocystine. Phytochemicals from orange sources fall within the families generally recognized as monoterpenes, terpenes and/or flavonoids. Examples of monoterpenes include limonene and d-limonene, typically found in orange peel oil.

The limonoid or limonoid glucoside group of the terpene family includes phytochemicals such as limonin or limonin glucoside from orange seeds, as well as nomilin. Others within this group of the terpene family are liminol, deoxyliminic acid, limonin carboxymethoxime, limonin-17-O-beta-d-glucoside, obacunone, obacunone-17-O-beta-d-glucoside, nomilin-17-O-beta-d-glucoside, deacetylnomilin, deacetylnomilin-17-O-beta-d-glucoside and deacetylnomilic-17O-beta-d-glucoside.

Included within the flavanones or flavanone glycosides group of the flavonoid family are the aglycones naringinin and hesperetin, as well as the glucosides naringin and hesperidin, or narirutin. Each of these flavanones is polyphenolic, and each is typically found in orange peel and juices. Additional flavanones are eriocitrin, didymin and poncitrin.

The methoxyflavone group of the flavonoid family also encompasses polyphenolic compounds. These methoxylated flavones include tangeretin and nobiletin. Other methoxyflavones include sinensetin, heptamethoxyflavone, tetra-O-methylscutellarein, and hexa-O-methylgossypetin.

Concerning these phytochemicals in orange juices, it is believed that the serum cholesterol modifying effective amount of the phytochemicals incorporated into the compositions can be considered in terms of levels typically found in orange sources. These effective amounts will vary depending upon the particular phytochemical. It will be generally understood that the quantity of phytochemical to be administered can be determined, for example, by assessing whether or not the particular phytochemical increases HDL serum cholesterol to a significant extent at the particular amount. Amounts or dosage levels can be expressed as a weight percent or on a parts per million basis. Dosages also can be expressed as weight of phytochemical per unit of body weight or blood serum volume.

A typical phytochemical dosage can be expressed as, for example, a specified level of milligrams per kilogram of body weight. Alternatively, and as used generally herein, dosages can be expressed as a certain quantity of phytochemical or essential human nutrient administered on a daily basis. A typical effective dosage level for a minor nutrient such as vitamin C could be expressed as, for example, 75 mg per day, while that for folate can be expressed as, for example, 63 μg per day, both typically as included in the treatment composition in combination with one or more phytochemicals.

Examples of daily effective dosages of the phytochemicals include the following. In the monoterpenes family, a typical limonene dosage is on the order of at least about 75 mg per day, preferably at least about 100 mg per day. In the terpene family, members of the limonoid glucoside group have a typical dosage level of at least about 75 mg per day, preferably at least about 150 mg per day. Of these, limonin glucoside has a dosage level of at least about 60 mg per day, preferably at least about 100 mg per day. A limonin level can be as low as about 1 mg per day. The flavanone glucoside group of the flavonoid family has a typical dosage level of at least about 100 mg per day. Of specific flavanone glucosides, hesperidin would have a typical treatment dosage of at least about 50 mg per day, and naringin would have a typical treatment dosage level of at least about 5 mg per day. Regarding the methoxyflavone group of the flavonoid family, the dosage level can be as low as about 1 mg per day.

Concerning these dosage levels, the levels can be considered to be either dosage levels of the sole component, or more typically, dosage levels when a multiplicity of these phytochemicals are incorporated within the treatment composition, either alone or in combination with one or more of the essential human nutrients.

The treatment methods according to the present invention are suitable for use in altering cholesterol levels of mammals or individuals, particularly of humans. Basically, the invention administers one or more of a soy component, a monoterpene, a terpene, and/or a flavonoid, preferably all in combination, at levels at which the beneficial cardiovascular health factor modification is achieved. These levels are generally exemplified hereinabove. Treatment times for achieving the cholesterol modifying effect typically will proceed with these dosage levels for several days to a few weeks as an initial effective dosage regimen.

Sources of whole orange components administered according to this procedure can include single strength juices, whether originating as a not from concentrate (NFC) juice or as a from concentrate (FC) juice. NFC juices are preferred and can have the benefit of enhanced effectiveness in the administration procedure of the invention. These orange juices can be fortified with extra levels of components such as vitamin C, vitamin E and so forth. Whatever the orange juice source and its make up, the compositions administered are soy fortified.

An important aspect of the invention is that the soy fortification be achieved with a soy protein source which has a make-up that enhances its solubility within orange juice and without detrimentally affecting sensory attributes in any substantial and unacceptable way. With the compositions administered according to the invention, the soy protein source remains in solution in the orange juice for a time adequate for a commercially distributed orange juice. For a liquid whole juice packaged in consumer-sized cartons or containers, this time can be for several weeks, up to six months. The soy protein source has very high solubility in orange juices, which typically have a pH of between about 3.2 to about 4.4, without requiring heating or high shear mixing that are typically are detrimental to a juice.

The soy protein source as incorporated into the treatment composition also has a very low color impact on the orange juice color and adds no sensorally detectible grittiness to the juice, which substantially maintains the mouth feel and sensorally perceptible viscosity of the orange juice prior to soy fortification. From a sensory perspective, the soy protein source does not impart a high bean note or vegetable flavor notes to the orange juice.

The soy protein source administered with orange juice is included in the administration composition at a level of at least about 0.1 weight percent and typically not greater than about 12.5 weight percent, each based upon the total weight of the treatment composition. Preferably, the soy protein source is present at a level between about 0.2 and about 8 weight percent, most preferably between about 0.3 and about 3 weight percent, all based upon the total weight of the treatment composition. FDA regulations permitting health claims for a product having 6.25 grams of soy protein per reference amount translate to a treatment formulation according to the invention at a level of about 2.9 weight percent, based on the total weight of the soy fortified orange juice composition.

A preferred soy protein source is a soy protein hydrolysate which has good solubility in acidic media. Suitable soy sources are characterized as having a short-chained peptide structure. A suitable soy source for the invention can be made according to the enzyme treatment of U.S. Pat. No. 6,221,423 (Cho, et al.) in which a soy protein isolate is contacted with an enzyme under particular conditions so as to hydrolyze the protein material into a peptide material containing peptides, the majority of which have a peptide chain length of 7 peptides or less. The subject matter of this patent explicitly is incorporated by reference hereinto.

The soy fortified juice compositions reduce LDL to HDL cholesterol ratios by at least 0.1, often by at least 0.3 or at least 0.5. Systolic blood pressure reductions are at levels of at least about 2 mmHg.

The following Examples provide illustrations of the disclosure herein.

### EXAMPLE 1

Soy protein was incorporated into single strength orange juice, not from concentrate orange juice, and pasteurized in accordance with standard industry practice. The soy protein was provided by Protein Technologies, Inc. under #E99969-89-5. It was incorporated at a level of 0.97 weight percent, based upon the total weight of the treatment composition, to achieve a product having 2 grams per 8 ounce serving of soy fortified orange juice treatment composition. The dry powder soy protein was incorporated with high shear mixing for only 10 to 15 seconds and without heating.

The resulting soy fortified orange juice product showed that the soy fortification had only low color impact on the orange juice coloration, imparted only a very low bean flavor to the juice, gave no grittiness, and added no thickness or viscosity to the orange juice. No high off-flavor notes (beany or vegetable flavor) were detected. The nature of the soy protein allowed incorporation of nutritionally significant levels of soy protein to be incorporated into this orange juice without significant changes to the sensory qualities of the unfortified juice.

The fortified orange juice was administered to human subjects in daily doses.

### EXAMPLE 2

Soy protein was incorporated into single strength orange juice. The soy protein was provided by Protein Technologies, Inc. under #E99969-89-5. It was incorporated at a level of 0.465 weight percent, based upon the total weight of the treatment composition, to achieve a product having 1 gram per 8 ounce serving of soy fortified orange juice treatment composition. The dry powder soy protein was readily incorporated without heating.

The resulting soy fortified orange juice product showed that the soy fortification had virtually no color impact on the orange juice coloration, imparted no readily decectable bean flavor to the juice, gave no grittiness, and added no thickness or viscosity to the orange juice. The soy fortified orange juice was administered to human subjects in daily doses.

It will be understood that the embodiments of the present invention which have been described are illustrative of some of the applications of the principles of the present invention. Numerous modifications may be made by those skilled in the art without departing from the true spirit and scope of the invention.

1. A method of treating individuals to favorably modify cardiovascular health risk indicators in the individuals, which comprises having the individual consume an effective concentration of a soy fortified orange juice treatment composition comprising single strength orange juice and soy protein, wherein said soy protein is at a level of at least about 0.1 weight percent, and wherein said effective concentration modifies cholesterol levels of the individual in at least one health-enhancing manner.

2. The method in accordance with claim 1, wherein said health-enhancing manner raises the HDL cholesterol level of the individual.

3. The method in accordance with claim 1, wherein said health-enhancing manner lowers the LDL cholesterol level of the individual.

4. The method in accordance with claim 1, wherein said health-enhancing manner lowers the LDL to HDL cholesterol ratio of the individual by at least 0.1.

5. The method in accordance with claim 1, wherein said soy fortified orange juice treatment composition includes a nutrient selected from the group consisting of a folate, iron, potassium, B vitamins, vitamin E and vitamin C.

6. The method in accordance with claim 1, wherein said effective concentration is about 8 ounces of the soy fortified orange juice treatment composition, and wherein said composition is administered daily to the individual.

7. The method in accordance with claim 1, wherein said effective concentration is at least about 15 ounces of the soy fortified orange juice treatment composition, and wherein said composition is administered daily to the individual.

8. The method in accordance with claim 1, wherein said soy protein is present at a level of not greater than about 12.5 weight percent, based on the total weight of the soy fortified orange juice treatment composition.

9. The method in accordance with claim 1, wherein said orange juice has a pH of between about 3.2 and about 4.4.

10. The method in accordance with claim 1, wherein said soy protein is a soy protein hydrolysate having a short-chained peptide structure.

11. The method in accordance with claim 1, wherein said effective concentration of the soy fortified orange juice treatment composition lowers at least one blood pressure reading of the individual.

12. The method in accordance with claim 11, wherein the systolic blood pressure level of the individual is reduced by at least about 2 mmHg.

13. The method in accordance with claim 4, wherein said LDL to HDL ratio is decreased by at least 0.3.

14. The method in accordance with claim 4, wherein said LDL to HDL ratio is decreased by at least 0.5.

15. A method of treating individuals to favorably modify cardiovascular health risk indicators in the individuals, which comprises having an individual consume an effective concentration of about 8 ounces of a soy fortified orange juice treatment composition per day, the composition comprising single strength orange juice and soy protein, wherein said soy protein is at a level of at least about 0.2 weight percent, based upon the total weight of the composition, and wherein said effective concentration modifies cholesterol levels of the individual in at least one health-enhancing manner.

16. The method in accordance with claim 15, wherein said health-enhancing manner raises the HDL cholesterol level of the individual and also lowers blood pressure of the individual.

17. The method in accordance with claim 1, wherein said treatment composition includes ingredients with nutritional value which are in addition to those found in the juice or soy.

18. The method in accordance with claim 1, wherein said health-enhancing manner lowers the total cholesterol level.
